(19) United States
(12) Patent Application Publication
(10) Pub. No.: US 2020/0354293 A1
(43) Pub. Date: Nov. 12, 2020

(54) PROCESS AND APPARATUS FOR DUAL FEED PARA-XYLENE SEPARATION

(71) Applicant: UOP LLC, Des Plaines, IL (US)

(72) Inventors: Robert E. Tsai, Arlington Heights, IL (US); Joseph Montalbano, Elmhurst, IL (US); Ellen Arnold, Wheeling, IL (US); Raymond Shih, Elgin, IL (US); Anton N. Mlinar, Vernon Hills, IL (US)

(21) Appl. No.: 16/408,807

(22) Filed: May 10, 2019

(51) Int. Cl. C07C 7/09; C07C 7/00; C07C 2/76; C07C 7/12

(52) U.S. Cl. CPC C07C 7/09; C07C 7/005; B01D 3/14; C07C 7/12; C07C 2/76

(57) ABSTRACT

Processes for recovering para-xylene from a para-xylene separation zone that receives two or more feed streams with different para-xylene amounts. One of the feed streams may have an equilibrium amount while the second may have a high amount. A fractionation column provides the effluent stream with para-xylene which may be a high purity stream that can be fed to the para-xylene separation zone in its entirety. If heavier compounds need to be removed from the second feed stream, a split shell or divided wall column may be used.

### FIELD OF THE INVENTION

The present invention relates to a process and apparatus for dual feed para-xylene separation.

### BACKGROUND OF THE INVENTION

Aromatics, particularly benzene, toluene, ethylbenzene, and the xylenes (ortho, meta, and para isomers), which are commonly referred to as “BTEX” or more simply “BTX,” are extremely useful chemicals in the petrochemical industry. They represent the building blocks for materials such as polystyrene, styrene-butadiene rubber, polyethylene terephthalate, polyester, phthalic anhydride, solvents, polyurethane, benzoic acid, and numerous other components. Conventionally, BTEX is obtained for the petrochemical industry by separation and processing of fossil-fuel petroleum fractions, for example, in catalytic reforming or cracking refinery process units. The different aromatic compounds can be separated from each in an aromatic complex which has various separation units, as well as processing units for increasing the recovery of specific compounds.

Specifically, para-xylene and meta-xylene are important raw materials in the chemical and fiber industries. Terephthalic acid derived from para-xylene is used to produce polyester fabrics and other articles which are in wide use today. One or a combination of adsorptive separation, crystallization and fractional distillation have been used to obtain these xylene isomers, with adsorptive separation capturing a great majority of the market share of newly constructed plants for the dominant para-xylene isomer.

Processes for adsorptive separation are widely described in the literature. For example, a general description directed to the recovery of para-xylene was presented at page 70 of the September 1970 edition of Chemical Engineering Progress (Vol. 66, No 9). There is a long history of available references describing useful adsorbents and desorbents, mechanical parts of a simulated moving-bed system including rotary valves for distributing liquid flows, the internals of the adsorbent chambers and control systems.

The para-xylene separation unit in a BTX complex is responsible for recovering the key high-purity para-xylene product. The feed to the unit typically comes from the xylene fractionation column and comprises an equilibrium mixture of xylenes (i.e., 23% para-xylene). The feed is processed through numerous adsorbent zones, and the resulting extract is fractionated to separate out the para-xylene. The para-xylene separation unit requires significant capital and utilities and is typically a principal cost within the aromatics complex.

While a feed stream to the aromatic complex may typically have an equilibrium amount of the xylene isomers, the aromatic complex may also produce or provide a mixed xylene stream that has a different, specifically, much greater amount of, for example, para-xylene. For example, toluene methylation alkylates toluene using methanol to produce para-xylene at very high (90+% para-xylene) selectivity relative to total xylenes.

It would be desirable to have processes which effectively and efficiently processes the two different feeds to the para-xylene separation unit which have different amounts of para-xylene.

### SUMMARY OF THE INVENTION

One or more processes for the separation and recovery of para-xylene from a para-xylene separation unit receiving feeds with different levels of para-xylene in the streams have been invented. Generally, in the present processes, two feed streams are sent to the para-xylene separation unit (but more than two may be included). The first feed stream is a typical para-xylene separation unit feed featuring an equilibrium (23%) concentration of para-xylene, for example, produced via combination of an A8 stripper sidedraw and an A8 rerun column distillate streams. The second feed stream features a high (90+%) concentration of para-xylene. For example, the high purity para-xylene stream may be derived from a toluene methylation unit and may be routed to the para-xylene separation unit in different ways.

Specifically, the toluene methylation unit product fractionator may be operated to produce a pure para-xylene sidedraw, the entirety of which can be sent to the para-xylene separation unit. Alternatively, the toluene methylation unit product fractionator may be operated to send xylenes and heavier aromatics to its bottoms stream. This bottoms stream can then be processed in a split-shell rerun column that has a top dividing wall, effectively splitting an equilibrium-purity para-xylene feed from the high-purity para-xylene feed, enabling removal of heavy aromatics from the toluene methylation unit product fractionator bottoms stream while maintaining its high para-xylene concentration.

Therefore, the present invention may be characterized, in at least one aspect, as providing a process for recovering para-xylene by: separating a feed into at least a first stream comprising mixed xylenes, and a toluene stream comprising toluene; feeding, into a para-xylene separation zone, at least a portion of the first stream; alkylating, in a toluene methylation zone, toluene from the toluene stream with methanol to provide a methylated effluent comprising para-xylene; separating, in a fractionation column, the methylated effluent into an effluent stream comprising para-xylene; feeding, into the para-xylene separation zone, at least a portion of the effluent stream comprising para-xylene; and, recovering a para-xylene product stream from the para-xylene separation zone.

The present invention may also be generally characterized, in at least one aspect, as providing a process for recovering para-xylene by: separating a feed into at least a first stream comprising mixed xylenes, and a toluene stream comprising toluene; feeding, into a para-xylene separation zone, at least a portion of the first stream; alkylating, in a toluene methylation zone, toluene from the toluene stream with methanol to provide a methylated effluent comprising para-xylene; separating, in a fractionation column, the methylated effluent into an overhead stream, a sidedraw stream, and a methylated effluent bottoms stream, and wherein the sidedraw stream comprises para-xylene; feeding, into the para-xylene separation zone, all of the sidedraw stream from the fractionation column; and, recovering a para-xylene product stream from the para-xylene separation zone.

The present invention may further be characterized, in at least one aspect, as providing a process for recovering para-xylene by: separating a feed into at least a first stream comprising mixed xylenes, and a toluene stream comprising toluene; feeding, into a para-xylene separation zone, at least a portion of the first stream; alkylating, in a toluene methylation zone, toluene from the toluene stream with methanol to provide a methylated effluent comprising para-xylene; separating, in a fractionation column, the methylated effluent into an overhead stream and a methylated effluent bottoms stream, and wherein the methylated effluent bottoms stream comprises para-xylene; feeding, into the para-xylene separation zone, only a portion of the methylated effluent bottoms stream; and, recovering a para-xylene product stream from the para-xylene separation zone.

Additional aspects, embodiments, and details of the invention, all of which may be combinable in any manner, are set forth in the following detailed description of the invention.

### Definitions

As used herein, the term “stream”, “feed”, “product”, “part” or “portion” can include various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. Each of the above may also include aromatic and non-aromatic hydrocarbons.

Hydrocarbon molecules may be abbreviated C₁, C₂, C₃, C. where “n” represents the number of carbon atoms in the one or more hydrocarbon molecules or the abbreviation may be used as an adjective for, e.g., non-aromatics or compounds. Similarly, aromatic compounds may be abbreviated A₆, A₇, A₈, An where “n” represents the number of carbon atoms in the one or more aromatic molecules. Furthermore, a superscript “+” or “−” may be used with an abbreviated one or more hydrocarbons notation, e.g., C₃₊ or C₃₋, which is inclusive of the abbreviated one or more hydrocarbons. As an example, the abbreviation “C₃₊” means one or more hydrocarbon molecules of three or more carbon atoms.

As used herein, the term “zone” can refer to an area including one or more equipment items and/or one or more sub-zones. Equipment items can include, but are not limited to, one or more reactors or reactor vessels, separation vessels, distillation towers, heaters, exchangers, pipes, pumps, compressors, and controllers. Additionally, an equipment item, such as a reactor, dryer, or vessel, can further include one or more zones or sub-zones.

As used herein, the term “rich” can mean an amount of at least generally 50%, and preferably 70%, by mole, of a compound or class of compounds in a stream.

### DETAILED DESCRIPTION OF THE DRAWINGS

One or more exemplary embodiments of the present invention will be described below in conjunction with the following drawing figures, in which:

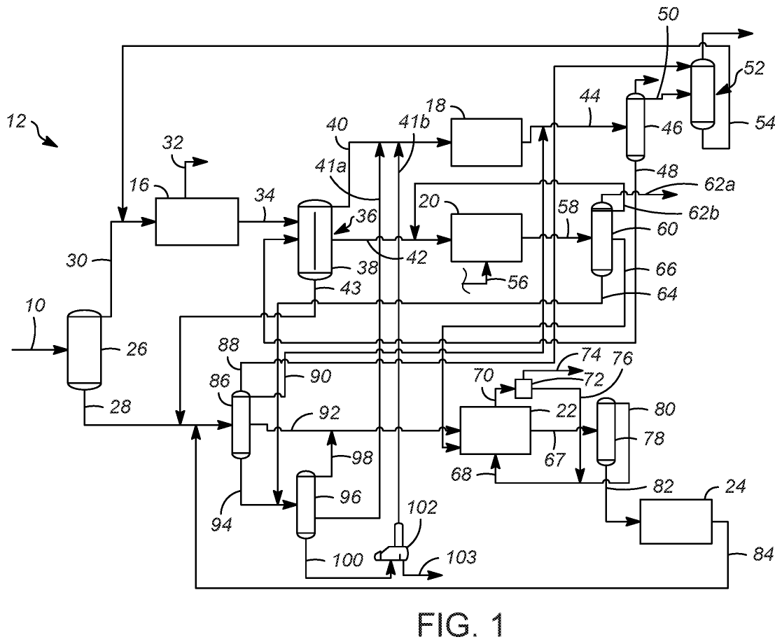

FIG. 1 shows a process flow diagram according to one aspect of the present invention; and,

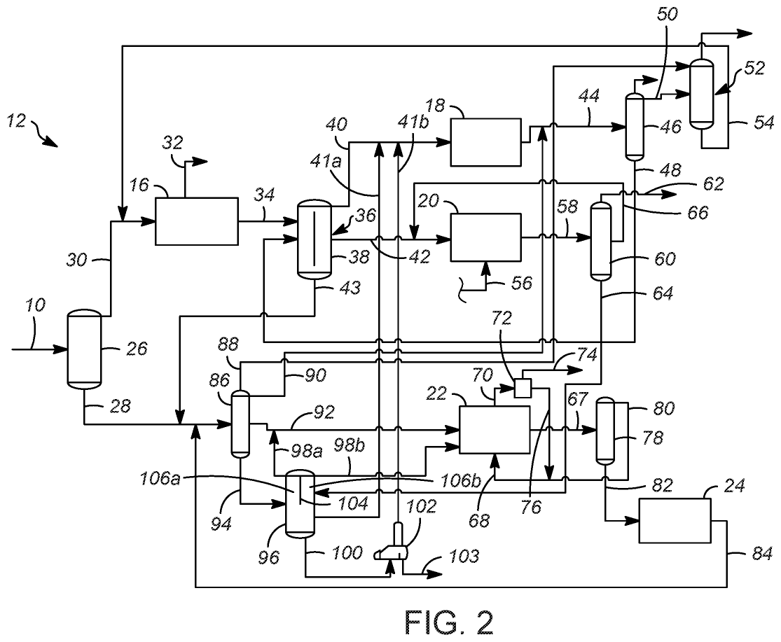

FIG. 2 shows a process flow diagram according to another aspect of the present invention.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present disclosure. Also, common but well-understood elements that are useful or necessary in a commercially feasible embodiment are often not depicted in order to facilitate a less obstructed view of these various embodiments of the present disclosure.

### DETAILED DESCRIPTION OF THE INVENTION

As mentioned above, processes have been invented which effectively and efficiently recover para-xylene from a para-xylene separation unit which receives two different para-xylene feeds, with different amounts of para-xylene. With these general principles in mind, one or more embodiments of the present invention will be described with the understanding that the following description is not intended to be limiting.

In FIGS. 1 and 2, an aromatics-containing feed stream 10 to an aromatic complex 12 generally comprises alkylaromatic hydrocarbons of the general formula C₆H₍₆₋ₙ₎Rₙ, where n is an integer from 0 to 5 and each R may be CH₃, C₂H₅, C₃H₇, or C₄H₉, in any combination. The aromatics-containing feed stream 10 contains benzene, toluene and aromatics and typically contains higher aromatics and aliphatic hydrocarbons including naphthenes.

The feed stream 10 to the process of the present disclosure may be derived from a variety of sources, including without limitation catalytic reforming, steam pyrolysis of naphtha, distillates or other hydrocarbons to yield light olefins and heavier aromatics-rich byproducts (including gasoline-range material often referred to as “pygas”), and catalytic or thermal cracking of distillates and heavy oils to yield products in the gasoline range. Products from pyrolysis or other cracking operations generally will be hydrotreated according to processes well known in the industry before being charged to the complex in order to remove sulfur, olefins and other compounds which would affect product quality and/or damage catalysts or adsorbents employed therein. Light cycle oil from catalytic cracking also may be beneficially hydrotreated and/or hydrocracked according to known technology to yield products in the gasoline range; the hydrotreating preferably also includes catalytic reforming to yield the aromatics-rich feed stream. The reforming zone, as is known, generally includes a reforming unit that receives a feed. The reforming unit will typically comprise a reforming catalyst. Usually such a stream will also be treated to remove olefinic compounds and light ends, e.g., butanes and lighter hydrocarbons and preferably pentanes, such removal, however, is not essential to the practice of the broad aspects of this disclosure and is not shown.

As shown in FIGS. 1 and 2, the aromatics complex 12 includes, for example, an aromatics extraction zone 16, transalkylation zone 18, a toluene methylation zone 20, and para-xylene separation zone 22, and a xylene isomerization zone 24.

In the depicted embodiments, the feed stream 10 is separated in a fractionation column 26, such as a reformate splitter. The reformate splitter functions to separate or “split” the aromatics-containing feed stream 10 by distilling the aromatics-containing feed stream 10 into a heavier, higher boiling fraction as stream 28 and a lighter, lower boiling fraction as stream 30. The fractionation column 26 may be configured such that, for example, the heavier fraction in stream 28 includes primarily, such as greater than about 80%, greater than about 90%, or greater than about 95%, hydrocarbons having eight or more carbon atoms (C₈₊). The lighter fraction in stream 30 may include primarily (such as greater than about 80%, greater than about 90%, or greater than about 95%) hydrocarbons having seven or fewer carbon atoms (C₇₋) and including benzene and toluene from the feed stream 10.

The lighter fraction in stream 30 is passed to the aromatics extraction zone 16 which may be, for example, an extractive distillation process unit which separates a largely aliphatic raffinate in stream 32 from a benzene-toluene aromatics stream 34. The further processing of the aliphatic raffinate in stream 32 is not necessary for practicing or understanding the present invention. The benzene-toluene aromatics stream 34 is passed to a fractionation zone 36 having one or more fractionation columns 38 which separate the components and produce at least one benzene stream 40, one toluene stream 42, and a stream 43 with heavier hydrocarbons, including xylene (discussed below). The depicted fractionation zone 36 includes a single fractionation column 38, which is a split shell fractionation column. Other configurations are contemplated.

The benzene stream 40, along with one or more heavy aromatics streams 41a, 41b (discussed below), is passed to the transalkylation zone 18. In one embodiment, the transalkylation zone 18 includes at least one reactor with conditions including a temperature of 320 to 440° C. (608 to 824° F.). The transalkylation zone may contain a first catalyst. In one embodiment, the first catalyst comprises at least one zeolitic component suitable for transalkylation, at least one zeolitic component suitable for dealkylation and at least one metal component suitable for hydrogenation. Such catalyst and conditions for the transalkylation zone 18 are known in the art.

A transalkylation effluent stream 44 is passed to a transalkylation stripper column 46 which provides a bottoms transalkylation effluent 48, including benzene, toluene, and heavier compounds, which is returned to the fractionation zone 36. A transalkylation effluent overhead 50 may be passed to a stabilization zone 52 which provides a transalkylation stabilized bottoms stream 54, which may include benzene, and which is passed back to the aromatics extraction zone 16.

Returning to the fractionation zone 36, the toluene stream 42, along with a methanol stream 56, is passed to the toluene methylation zone 20 which includes a reactor and is operated under condition, as are known, so that the toluene is alkylated with the methanol to produce xylenes. A toluene methylation effluent stream 58 is passed to a fractionation column 60 which separates the toluene methylation effluent stream 58 into at least one effluent stream comprising para-xylene. The fractionation column 60 may be operated to obtain different streams associated with the separation of toluene from the xylenes in the toluene methylation effluent stream 58, including a high purity para-xylene stream. According to the embodiment of FIG. 1, the fractionation column 60 is operated so that it provides two overhead streams 62a, 62b, a bottoms stream 64, and a sidedraw stream 66 that is the effluent stream comprising para-xylene. The heavier overhead stream 62b may be recycled to the toluene methylation zone 20.

The sidedraw stream 66 has a non-equilibrium mixture of xylenes, specifically having a higher concentration of para-xylene compared with the streams in the complex having an equilibrium mixture of xylenes. According to the present invention, the entirety of the sidedraw stream 66 is fed to the para-xylene separation zone 22.

The para-xylene separation zone 22 preferably includes an extraction unit that utilizes adsorption to, as is known in the art, selectively adsorb para-xylene while providing a raffinate stream 67. A desorbent stream 68 is used to desorb the para-xylene from the adsorbent in an extract stream 70. The extract stream 70 is separated in an extract column 72 into a para-xylene product stream 74, and a desorbent stream 76. The raffinate stream 67 is separated in a raffinate column 78 into a second desorbent stream 80 which may be combined with the and desorbent stream 76 and used as the desorbent stream 68. The raffinate column 78 also provides a para-xylene lean xylene stream 82.

The para-xylene lean xylene stream 82 comprising a non-equilibrium mixture of xylene isomers and ethylbenzene may be passed to the xylene isomerization zone 24. The xylene isomerization zone 24 includes a reactor which contains an isomerization catalyst and is operated under conditions to isomerize the xylenes and provide a product approaching equilibrium concentrations of C₈-aromatic isomers. In one embodiment, the isomerization conditions include a temperature of about 240 to about 440° C. (464 to 824° F.). Further, the xylene isomerization zone 24 may include a second catalyst. In one embodiment, the second catalyst comprises at least one zeolitic component suitable for xylene isomerization, at least one zeolitic component suitable for ethylbenzene conversion, and at least one metal component suitable for hydrogenation. In one embodiment, the isomerization process is carried out in the vapor phase. In yet another embodiment, the isomerization process is carried out in the liquid phase. In one embodiment, the isomerization process converts ethylbenzene by dealkylation to produce benzene. In another embodiment, the isomerization process converts ethylbenzene by isomerization to produce xylenes.

A xylene isomerization effluent stream 84, along with the heavier fraction 28 from the fractionation column 26, and the stream 43 with heavier hydrocarbons, including xylene, from the fractionation zone 36, are passed to a stripper column 86. A first stripper overhead stream 88 may be passed to the stabilization zone 52 discussed above. A second stripper overhead stream 90, with heavier hydrocarbons than the first stripper overhead stream 88 may be combined with the transalkylation effluent stream 44, also discussed above. A sidedraw stream 92 from the stripper column 86 comprises an equilibrium mixture of xylenes and is passed to the para-xylene separation zone 22. Although depicted as being fed to the para-xylene separation zone 22 separate from the other feed stream (sidedraw stream 66 from the fractionation column 60), it is contemplated (for any of the embodiments herein) that the two feeds are combined prior to being fed into the separation unit of the para-xylene separation zone 22 or they may be segregated and fed separately. The further processing of the components of this stream is the same as discussed above.

A stripper bottoms stream 94 from the stripper column 86, along with the bottoms stream 64 from the fractionation column 60, may be passed to a rerun column 96. A rerun overhead stream 98 including xylenes may be combined with the sidedraw stream 92 from the stripper column 86. The rerun column 96 may produce a sidedraw stream that is one of the heavy aromatic streams 41a that is passed to the transalkylation zone 18. A rerun bottoms stream 100 that is passed to a heavy aromatic rectifier 102, which may provide another one of the heavy aromatic streams 41b passed to the transalkylation zone 18. The further processing of the heavy aromatics portion 103 is not necessary for the practicing or understanding of the present invention.

Turning to FIG. 2, the fractionation column 60 is not operated to provide a high purity para-xylene sidedraw stream, rather the effluent stream comprising para-xylene from the fractionation column 60 is the bottoms stream 64. Accordingly, before the para-xylene can be recovered in the para-xylene separation zone 22, the other components in the bottoms stream 64 from the fractionation column 60 must be separated.

Therefore, the bottoms stream 64 from the fractionation column 60 is passed to a rerun column 96′. The rerun column 96′ of FIG. 2 is a divided wall column that includes a wall 104 that separates the middle and upper portions of the rerun column 96′ into two sections or separation zones 106a, 106b. The first separation zone 106a receives the stripper bottoms stream 94 from the stripper column 86 and provides a first overhead stream 98a having an equilibrium mixture of xylenes. The first overhead stream 98a stream may be combined with the sidedraw stream 92 from the stripper column 86 and passed to the para-xylene separation zone 22. The second separation zone 106b receives the bottoms stream 64 from the fractionation column 60 and provides a second overhead stream 98b having a non-equilibrium mixture of xylenes (specifically a much higher amount of para-xylene). The second overhead stream 98b is fed to the para-xylene separation zone 22. The bottom portions of the two separation zones 106a, 106b may be combined so that the rerun column 96′ provides a single, or common, bottoms stream 100. Finally, the sidedraw stream 66 from the fractionation column 60 is recycled to the toluene methylation zone 20.

In the embodiment of FIG. 2, a lower energy separation may be used in the fractionation column 60 to provide the effluent stream with para-xylene.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understanding the embodiments of the present invention.

Any of the above lines, conduits, units, devices, vessels, surrounding environments, zones or similar may be equipped with one or more monitoring components including sensors, measurement devices, data capture devices or data transmission devices. Signals, process or status measurements, and data from monitoring components may be used to monitor conditions in, around, and on process equipment. Signals, measurements, and/or data generated or recorded by monitoring components may be collected, processed, and/or transmitted through one or more networks or connections that may be private or public, general or specific, direct or indirect, wired or wireless, encrypted or not encrypted, and/or combination(s) thereof; the specification is not intended to be limiting in this respect.

Signals, measurements, and/or data generated or recorded by monitoring components may be transmitted to one or more computing devices or systems. Computing devices or systems may include at least one processor and memory storing computer-readable instructions that, when executed by the at least one processor, cause the one or more computing devices to perform a process that may include one or more steps. For example, the one or more computing devices may be configured to receive, from one or more monitoring component, data related to at least one piece of equipment associated with the process. The one or more computing devices or systems may be configured to analyze the data. Based on analyzing the data, the one or more computing devices or systems may be configured to determine one or more recommended adjustments to one or more parameters of one or more processes described herein. The one or more computing devices or systems may be configured to transmit encrypted or unencrypted data that includes the one or more recommended adjustments to the one or more parameters of the one or more processes described herein.

### Specific Embodiments

While the following is described in conjunction with specific embodiments, it will be understood that this description is intended to illustrate and not limit the scope of the preceding description and the appended claims.

A first embodiment of the invention is a process for recovering para-xylene, the process comprising separating a feed into at least a first stream comprising mixed xylenes, and a toluene stream comprising toluene; feeding, into a para-xylene separation zone, at least a portion of the first stream; alkylating, in a toluene methylation zone, toluene from the toluene stream with methanol to provide a methylated effluent comprising para-xylene; separating, in a fractionation column, the methylated effluent into an effluent stream comprising para-xylene; feeding, into the para-xylene separation zone, at least a portion of the effluent stream comprising para-xylene; and, recovering a para-xylene product stream from the para-xylene separation zone. An embodiment of the invention is one, any or all of prior embodiments in this paragraph up through the first embodiment in this paragraph, wherein the fractionation column provides an overhead stream, a sidedraw stream, and a methylated effluent bottoms stream, and wherein the sidedraw stream comprises the effluent stream comprising para-xylene from the fractionation column. An embodiment of the invention is one, any or all of prior embodiments in this paragraph up through the first embodiment in this paragraph, wherein all of the sidedraw stream is fed to the para-xylene separation zone. An embodiment of the invention is one, any or all of prior embodiments in this paragraph up through the first embodiment in this paragraph, further comprising combining the methylated effluent bottoms stream with a portion of the first stream. An embodiment of the invention is one, any or all of prior embodiments in this paragraph up through the first embodiment in this paragraph, further comprising separating, in a stripper column, a mixed xylene stream from the first stream. An embodiment of the invention is one, any or all of prior embodiments in this paragraph up through the first embodiment in this paragraph, wherein the mixed xylene stream comprises a stripper bottoms stream further comprising heavy aromatics, and wherein the process further includes separating, in a rerun column, the stripper bottoms stream into at least a rerun overhead stream and a rerun bottoms stream, wherein the rerun overhead stream is fed into the para-xylene separation zone. An embodiment of the invention is one, any or all of prior embodiments in this paragraph up through the first embodiment in this paragraph, further comprising separating, in the rerun column with the stripper bottoms stream, the methylated effluent bottoms stream. An embodiment of the invention is one, any or all of prior embodiments in this paragraph up through the first embodiment in this paragraph, wherein the fractionation column provides an overhead stream and a methylated effluent bottoms stream, and wherein the methylated effluent bottoms stream comprises the effluent stream comprising para-xylene. An embodiment of the invention is one, any or all of prior embodiments in this paragraph up through the first embodiment in this paragraph, further comprising separating, in a stripper column, a mixed xylene stream from the first stream, wherein the mixed xylene stream comprises a stripper bottoms stream. An embodiment of the invention is one, any or all of prior embodiments in this paragraph up through the first embodiment in this paragraph further comprising separating, in a rerun column, the stripper bottoms stream into at least a first rerun overhead stream and a rerun bottoms stream, wherein the first rerun overhead stream is fed into the para-xylene separation zone; and, separating, in the rerun column, the methylated effluent bottoms stream into at least a second rerun overhead stream comprising para-xylene, the second rerun overhead stream comprising a greater purity of para-xylene than the first rerun overhead stream, wherein the second rerun overhead stream comprises the portion of the effluent stream comprising para-xylene fed into the para-xylene separation zone. An embodiment of the invention is one, any or all of prior embodiments in this paragraph up through the first embodiment in this paragraph, wherein the rerun column comprises a wall separating the rerun column into a first separation zone and a second separation zone, and wherein the first separation zone receives the stripper bottoms stream and provides the first rerun overhead stream and the second separation zone receives the methylated effluent bottoms stream and provides the second rerun overhead stream. An embodiment of the invention is one, any or all of prior embodiments in this paragraph up through the first embodiment in this paragraph, wherein the first separation zone and the second separation zone are combined at a bottom of the rerun column.

A second embodiment of the invention is a process for recovering para-xylene, the process comprising separating a feed into at least a first stream comprising mixed xylenes, and a toluene stream comprising toluene; feeding, into a para-xylene separation zone, at least a portion of the first stream; alkylating, in a toluene methylation zone, toluene from the toluene stream with methanol to provide a methylated effluent comprising para-xylene; separating, in a fractionation column, the methylated effluent into an overhead stream, a sidedraw stream, and a methylated effluent bottoms stream, and wherein the sidedraw stream comprises para-xylene; feeding, into the para-xylene separation zone, all of the sidedraw stream from the fractionation column; and, recovering a para-xylene product stream from the para-xylene separation zone. An embodiment of the invention is one, any or all of prior embodiments in this paragraph up through the second embodiment in this paragraph, further comprising separating, in a stripper column, a mixed xylene stream from the first stream, wherein the mixed xylene stream comprises a stripper bottoms stream. An embodiment of the invention is one, any or all of prior embodiments in this paragraph up through the second embodiment in this paragraph, wherein the stripper bottoms stream further comprises heavy aromatics, and wherein the processing further includes separating, in a rerun column, the stripper bottoms stream into at least a rerun overhead stream and a rerun bottoms stream, wherein the rerun overhead stream is fed into the para-xylene separation zone. An embodiment of the invention is one, any or all of prior embodiments in this paragraph up through the second embodiment in this paragraph, further comprising separating, in the rerun column with the stripper bottoms stream, the methylated effluent bottoms stream.

A third embodiment of the invention is a process for recovering para-xylene, the process comprising separating a feed into at least a first stream comprising mixed xylenes, and a toluene stream comprising toluene; feeding, into a para-xylene separation zone, at least a portion of the first stream; alkylating, in a toluene methylation zone, toluene from the toluene stream with methanol to provide a methylated effluent comprising para-xylene; separating, in a fractionation column, the methylated effluent into an overhead stream and a methylated effluent bottoms stream, and wherein the methylated effluent bottoms stream comprises para-xylene; feeding, into the para-xylene separation zone, only a portion of the methylated effluent bottoms stream; and, recovering a para-xylene product stream from the para-xylene separation zone. An embodiment of the invention is one, any or all of prior embodiments in this paragraph up through the third embodiment in this paragraph further comprising separating, in a stripper column, a mixed xylene stream from the first stream, wherein the mixed xylene stream comprises a stripper bottoms stream; separating, in a rerun column, the stripper bottoms stream into at least a first rerun overhead stream and a rerun bottoms stream, wherein the first rerun overhead stream is fed into the para-xylene separation zone; and, separating, in the rerun column, the methylated effluent bottoms stream into at least a second rerun overhead stream comprising para-xylene, the second rerun overhead stream comprising a greater purity of para-xylene than the first rerun overhead stream, wherein the second rerun overhead stream comprises the portion of the methylated effluent bottoms stream from the fractionation column fed into the para-xylene separation zone. An embodiment of the invention is one, any or all of prior embodiments in this paragraph up through the third embodiment in this paragraph, wherein the rerun column comprises a wall separating the rerun column into a first separation zone and a second separation zone, and wherein the first separation zone receives the stripper bottoms stream and provides the first rerun overhead stream and the second separation zone receives the methylated effluent bottoms stream and provides the second rerun overhead stream. An embodiment of the invention is one, any or all of prior embodiments in this paragraph up through the third embodiment in this paragraph, wherein the first separation zone and the second separation zone are combined at a bottom of the rerun column.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. A process for recovering para-xylene, the process comprising:
separating a feed into at least a first stream comprising mixed xylenes, a benzene stream comprising benzene, and a toluene stream comprising toluene;
feeding, into a transalkylation zone, the benzene stream, feeding, into a para-xylene separation zone, at least a portion of the first stream;
alkylating, in a toluene methylation zone, toluene from the toluene stream with methanol to provide a methylated effluent comprising para-xylene;
separating, in a fractionation column, the methylated effluent into an effluent stream comprising para-xylene;
feeding, into the para-xylene separation zone, at least a portion of the effluent stream comprising para-xylene; and, recovering a para-xylene product stream from the para-xylene separation zone.

2. The process of claim 1, wherein the fractionation column provides an overhead stream, a sidedraw stream, and a methylated effluent bottoms stream, and wherein the sidedraw stream comprises the effluent stream comprising para-xylene from the fractionation column.

3. The process of claim 2, wherein all of the sidedraw stream is fed to the para-xylene separation zone.

4. The process of claim 3, further comprising:
combining the methylated effluent bottoms stream with a portion of the first stream.

5. The process of claim 2, further comprising:
separating, in a stripper column, a mixed xylene stream from the first stream.

6. The process of claim 5, wherein the mixed xylene stream comprises a stripper bottoms stream further comprising C8+ aromatics, and wherein the process further includes:
separating, in a rerun column, the stripper bottoms stream into at least a rerun overhead stream and a rerun bottoms stream, wherein the rerun overhead stream is fed into the para-xylene separation zone.

7. The process of claim 6, further comprising:
separating, in the rerun column with the stripper bottoms stream, the methylated effluent bottoms stream.

8. The process of claim 1, wherein the fractionation column provides an overhead stream and a methylated effluent bottoms stream, and wherein the methylated effluent bottoms stream comprises the effluent stream comprising para-xylene.

9. The process of claim 8, further comprising:
separating, in a stripper column, a mixed xylene stream from the first stream, wherein the mixed xylene stream comprises a stripper bottoms stream.

10. The process of claim 9 further comprising:
separating, in a rerun column, the stripper bottoms stream into at least a first rerun overhead stream and a rerun bottoms stream, wherein the first rerun overhead stream is fed into the para-xylene separation zone; and,
separating, in the rerun column, the methylated effluent bottoms stream into at least a second rerun overhead stream comprising para-xylene, the second rerun overhead stream comprising a greater purity of para-xylene than the first rerun overhead stream,
wherein the second rerun overhead stream comprises the portion of the effluent stream comprising para-xylene fed into the para-xylene separation zone.

11. The process of claim 10, wherein the rerun column comprises a wall separating the rerun column into a first separation zone and a second separation zone, and wherein the first separation zone receives the stripper bottoms stream and provides the first rerun overhead stream and the second separation zone receives the methylated effluent bottoms stream and provides the second rerun overhead stream.

12. The process of claim 11, wherein the first separation zone and the second separation zone are combined at a bottom of the rerun column.

13. A process for recovering para-xylene, the process comprising:
separating a feed into at least a first stream comprising mixed xylenes, a benzene stream comprising benzene, and a toluene stream comprising toluene;
feeding, into a transalkylation zone, the benzene stream^{.},
feeding, into a para-xylene separation zone, at least a portion of the first stream;
alkylating, in a toluene methylation zone, toluene from the toluene stream with methanol to provide a methylated effluent comprising para-xylene;
separating, in a fractionation column, the methylated effluent into an overhead stream, a sidedraw stream, and a methylated effluent bottoms stream, and wherein the sidedraw stream comprises para-xylene;
feeding, into the para-xylene separation zone, all of the sidedraw stream from the fractionation column; and,
recovering a para-xylene product stream from the para-xylene separation zone.

14. The process of claim 13, further comprising:
separating, in a stripper column, a mixed xylene stream from the first stream, wherein the mixed xylene stream comprises a stripper bottoms stream.

15. The process of claim 14, wherein the stripper bottoms stream further comprises C8+ aromatics, and wherein the processing further includes:
separating, in a rerun column, the stripper bottoms stream into at least a rerun overhead stream and a rerun bottoms stream, wherein the rerun overhead stream is fed into the para-xylene separation zone.

16. The process of claim 15, further comprising:
separating, in the rerun column with the stripper bottoms stream, the methylated effluent bottoms stream.

17. A process for recovering para-xylene, the process comprising:
separating a feed into at least a first stream comprising mixed xylenes, a benzene stream comprising benzene, and a toluene stream comprising toluene;
feeding, into a transalkylation zone, the benzene stream^{.},
feeding, into a para-xylene separation zone, at least a portion of the first stream;
alkylating, in a toluene methylation zone, toluene from the toluene stream with methanol to provide a methylated effluent comprising para-xylene;
separating, in a fractionation column, the methylated effluent into an overhead stream and a methylated effluent bottoms stream, and wherein the methylated effluent bottoms stream comprises para-xylene;
feeding, into the para-xylene separation zone, only a portion of the methylated effluent bottoms stream; and,
recovering a para-xylene product stream from the para-xylene separation zone.

18. The process of claim 17 further comprising:
separating, in a stripper column, a mixed xylene stream from the first stream, wherein the mixed xylene stream comprises a stripper bottoms stream;
separating, in a rerun column, the stripper bottoms stream into at least a first rerun overhead stream and a rerun bottoms stream, wherein the first rerun overhead stream is fed into the para-xylene separation zone; and,
separating, in the rerun column, the methylated effluent bottoms stream into at least a second rerun overhead stream comprising para-xylene, the second rerun overhead stream comprising a greater purity of para-xylene than the first rerun overhead stream,
wherein the second rerun overhead stream comprises the portion of the methylated effluent bottoms stream from the fractionation column fed into the para-xylene separation zone.

19. The process of claim 18, wherein the rerun column comprises a wall separating the rerun column into a first separation zone and a second separation zone, and wherein the first separation zone receives the stripper bottoms stream and provides the first rerun overhead stream and the second separation zone receives the methylated effluent bottoms stream and provides the second rerun overhead stream.

20. The process of claim 19, wherein the first separation zone and the second separation zone are combined at a bottom of the rerun column.
